(19) United States
(12) Patent Application Publication
(10) Pub. No.: US 2006/0025717 A1
(43) Pub. Date: Feb. 2, 2006

(54) Method for forming hollow out-of-plane microneedles and devices formed hereby

(75) Inventors: Stefan Zimmermann, Stockelsdorf (DE); Boris Stoeber, Berkeley, CA (US); Dorian Liepmann, Lafayette, CA (US); Albert Pisano, Danville, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/117,641

(22) Filed: Apr. 27, 2005

Related U.S. Application Data
(63) Continuation-in-part of application No. 10/828,510, filed on Apr. 19, 2004.
(60) Provisional application No. 60/464,221, filed on Apr. 18, 2003.

(51) Int. Cl. A61M 31/00

(52) U.S. Cl. 604/46; 604/500; 264/164; 264/313

(57) ABSTRACT

A method and apparatus for forming microneedles and other microstructures using hardenable materials and useful for substance monitoring and/or drug delivery.

### CROSS REFERENCE TO RELATED APPLICATIONS

This application is a continuation-in-part of patent application Ser. No. 10/828,510 filed 19 Apr. 2004, which claims priority from provisional patent application 60/464,221, filed 18 Apr. 2003. These applications are incorporated herein by reference.

### STATEMENT AS TO RIGHTS TO INVENTIONS MADE UNDER FEDERALLY SPONSORED RESEARCH AND DEVELOPMENT

The Invention was made with government support under Grant (Contract) No. F30602-00-2-0566 awarded by the Department of Defense. The Government has certain rights to this invention.

### COPYRIGHT NOTICE

Pursuant to 37 C.F.R. 1.71(e), Applicants note that a portion of this disclosure contains material that is subject to copyright protection (such as, but not limited to, source code listings, screen shots, user interfaces, or user instructions, or any other aspects of this submission for which copyright protection is or may be available in any jurisdiction.). The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

Currently proposed systems for monitoring substances of interest, such as glucose, using small sampling and monitoring devices have a number of difficulties. For example, a microdialysis probe discussed for glucose monitoring in U.S. Pat. No. 6,091,976, Jul. 18, 2000 (M. Pfeiffer and U. Hoss) is a needle-type probe with dialysis fluid flowing in and out of the probe. The probe is inserted at a length of several millimeters underneath the skin at a shallow angle so that the probe stays in the epidermal tissue. A dialysis membrane separates the probe interior from the interstitial fluid surrounding the probe. This membrane allows diffusion of substances such as glucose from the interstitial fluid into the dialysis fluid flowing in and out of the probe. The interstitial fluid is not extracted. The dialysis fluid is then pumped past a sensor that can be placed downstream where the glucose level of the dialysis fluid is determined. The glucose concentration of the dialysis fluid has been found to correlate with the glucose level in the interstitial fluid.

Different proposals have been made for fabricating hollow microneedles (e.g., U.S. Pat. Nos. 5,591,139, 5,855,801, 5,855,801, 6,106,751; L. Lin, A. P. Pisano, and R. S. Muller, Proceedings of the 7^{th} International Conference on Solid-State Sensors and Actuators-Transducers '93, Yokohama, Japan, 1993, p. 237; K. S. Lebouitz, and A. P. Pisano, Proceedings Microstructures and Microfabrication Systems IV, Boston, Mass., 1998, p. 235, J. Chen, K. D. Wise, Technical Digest Solid-State Sensor and Actuator Workshop (Hilton Head Island, S.C., USA, 1994), p. 256; N. H. Talbot, A. P. Pisano, Technical Digest Solid-State Sensor and Actuator Workshop, Hilton Head Island, S. C., USA, 1998, p. 265; J. Brazzle, I. Papautsky, and A. B. Frazier, Proceedings of the 20^{th} Annual International Conference in Medicine and Biology Society, vol. 20, no. 4, p. 1837, 1998; J. Brazzle, D. Bartholomeusz, R. Davies, J. Andrade, R. A. Van Wagenen, and, A. B. Frazier, Technical Digest Solid-State Sensor and Actuator Workshop Hilton Head Island, S.C., USA, 2000, p. 199). These in-plane needle fabrication concepts aim at fabricating microneedles out of various materials using Micro Electromechanical System (MEMS) technology.

Proposals for hollow out-of-plane needles out of silicon include e.g., U.S. Pat. Nos. 6,406,638 B1, 6,533,949; B. Stoeber, and D. Liepmann; Fluid injection through out-of-plane microneedles, Proceedings of the 1^{st} Annual International IEEE-EMBS Special Topic Conference on Microtechnologies in Medicine and Biology, Lyon, France Oct. 12-Oct. 14 2000, pp. 224-228; P. Griss, G. Stemme, Novel, side opened out-of-plane microneedles for microfluidic transdermal interfacing, Proceedings of 15^{th} International Workshop on Micro Electro Mechanical Systems, Las Vegas, Nev., USA, pp. 467-470, 2002; J. G. E. Gardeniers, J. W. Berenschot, M. J. de Boer, Y. Yeshurun, M. Hefetz, R. van't Oever, and A. van den Berg, Proceedings of 15^{th} International Workshop on Micro Electro Mechanical Systems, Las Vegas, Nev., USA, 2002). Generally, these proposals can involve expensive fabrication steps, which can make these needles too expensive for many applications. In some proposals, solid microneedles have been used as a template for electroplating of thin metal structures (U.S. Pat. No. 6,334,856) to form arrays of metal microneedles. This fabrication process is potentially much cheaper compared to silicon needles, but the typically thin walls make these structures not rigid enough for many applications.

In a different attempt to reduce fabrication costs of microneedle arrays, solid needles have been proposed to be made out of polymers, which have been cast from a mold made using microfabrication technology (J. H. Park, S. Davis, Y. K. Yoon, M. R. Prausnitz, and M. G. Allen, Micromachined Biodegradable Microstructures, Proceedings of 16^{th} Annual International Conference on Micro Electro Mechanical Systems (MEMS), Kyoto, Japan, Jan. 19-Jan. 23 2003, pp. 371-374).

Other proposals include manufacturing needles from polymeric materials using, for example, phase separation techniques and an open mold (e.g., U.S. Patent Application 20040028875/PCT/NL01/00874 Dec. 3, 2001) or using sheets of deformable materials over pillars and molding techniques, etc., (e.g., U.S. Pat. No. 6,471,903, divisional of Ser. No. 09/328,946, filed Jun. 9, 1999.). Other proposals have involved using a compliant substrate and polymers to form needles (e.g., Joseph M. Bauer, T. A. Saif, and David J. Beebe, Surface Tension Driven Formation of Microstructures, Journal of Microelectro-mechanical Systems, Vol. 13, no. 4, August 2004, p. 553.)

None of these proposals, however, have demonstrated wide acceptance or ability to be easily manufactured or integrated with practical devices for monitoring and/or drug delivery.

### SUMMARY

The present invention, in specific embodiments, involves novels methods for minimally invasive substance monitoring or substance delivery, in particular using easy to manufacture hollow microneedles made from a formable and hardenable substance, such as a liquid, a powder, a mixture, a sublimatable solid, etc. In further embodiments, the invention provides a device and/or method for monitoring and/or delivering substances of interest, particularly substances in biological research and/or clinical settings. In further embodiments, the invention provides a device and/or method using out-of-plane microneedles fabricated from various hardenable substances to provide an improved integrated sensor or delivery device useful in various applications.

In specific embodiments, the invention provides methods for the fabrication of arrays of hollow out-of-plane microneedles out of materials that are generally in an initially fluidic, gaseous, or quasi-fluidic form such as curable polymers, polymer solutions, melts, mixtures, powders, suspensions, etc. In a preferred method, such a hardenable material is placed on a surface with thin perpendicular pillars. One or more different mechanisms as described below can be used to cause the material to be higher around the pillars than elsewhere on the surface to define the shape of the needles. In specific embodiments, pillars are removed after or during hardening leaving voids or passages and forming the needle lumens. A base plate used for forming the materials can also be removed, or can be incorporated with the needles and other components to form various substance delivery and/or monitoring systems.

In specific embodiments, such a hardenable material can be poured onto the molding surface from the top, or enter from one or more sides, or be pushed onto a substrate through bottom holes in the substrate. In other embodiments, a hardenable material can be condensed or sublimated onto pillars and a base-plate.

In specific embodiments, capillary action causes the formable material to rise up on the surface of the thin pillars, with the height of rise depending on the contact angle with the pillars, the surface tension of the hardenable material, and/or its specific weight. The hardenable material can then be cured, partially hardened or hardened in that conformation.

In other specific embodiments, reducing material volume can be used in conjunction with or instead of capillary action to generate a needle, with adhesion holding a hardenable material on the sidewall of the pillars while the overall level sinks. In other embodiments, materials can be employed that shrink as they harden and this volume reduction effect can be used to generate needle shapes. Similarly, reduction can be accomplished by evaporation of one or more constituents or of a reaction product of a hardenable mixture-or material.

In other embodiments where the hardenable material adheres well to the pillars, the pillars can be pulled up out of the material while allowing air, another material, or more material of the pillars to follow from the base, with the pulling action forming needle shaped structures around the pillars. Alternatively, a similar effect can be achieved by forcing a material, e.g., a solid, liquid or gaseous, from the flat supporting base plate up through a hardenable fluid.

In further embodiments, a material can be placed onto the pillars and base through condensation or sublimation. In this embodiment, the supporting plate may be kept at a different temperature from the pillars so that more material deposition occurs on the plate, while the material on the pillar is tapered due to a temperature gradient on the pillar surface along its axis.

Microneedles formed according to these specific embodiments of the invention avoid problems of some earlier microneedle proposals that involve relatively expensive fabrication procedures.

In more specific embodiments, the invention involves a method and/or apparatus for monitoring of substances in or delivering substances to interstitial fluid under the skin of a human or animal or under the outer layer of a plant using out-of-plane microneedles, particularly using a device with sufficient flexibility to assist in breaking the outer layer of skin. For humans and animals, this can allow painless everyday usage.

In other embodiments the invention relates generally to a method and apparatus for continuous monitoring of compounds in the epidermal interstitial fluid using devices manufactured according to one or more methods described herein. As a specific example, the invention relates to a minimally invasive method for sampling compounds from the epidermal interstitial fluid using hollow out-of-plane microneedles and the apparatus for sampling and analyzing these compounds. A particular application of this invention is to continuously monitor the epidermal interstitial fluid glucose level.

In further specific embodiments, the invention involves an array (used herein to indicate any type of grouping) of out-of-plane microneedle structures that penetrate a skin or other surface. In specific applications, the microneedles are approximately 200 μm long, which, for example, is sufficient to reach the epidermal interstitial fluid in humans, though substantially longer and/or substantially shorter microneedles can be used for a variety of human and other applications. In further embodiments, the invention involves microneedles that are pre-filled with a liquid, such as a buffer or delivery solution, resulting in a liquid-liquid interface between the liquid inside the needle and the interstitial fluid once the needle is inserted. Substances can be delivered to or diffuse from the interstitial fluid and the lumens of the out-of-plane microneedles. In further embodiments, a dialysis membrane is placed on an opposite side of a substrate from the microneedles. Thus, the membrane separates the needle lumens from the pre-filled fluid, which is pumped past the membrane to the sensor. The amount of a material of interest diffusing through the out-of-plane microneedles, through the membrane and into the dialysis fluid is generally defined by the total area where diffusion can take place. This area is defined by the total cross section of all needle lumens.

The present invention in specific embodiments provides a disposable sensor and/or delivery system that is minimally invasive and provides painless and easy application. An example of such a system consists of hollow out-of-plane microneedles formed from a hardenable substance to sample glucose or another substance from the interstitial fluid of the epidermis, an integrated dialysis membrane and an integrated electrochemical enzyme-based sensor. A different example of such a system consists of hollow out-of-plane microneedles to deliver drugs or other substances to the interstitial fluid of the epidermis or deeper, below the epidermis.

In a further and very specific example embodiment, an array of between about 600 to 1500 microneedles formed from a hardenable substance is placed on an approximately 8 mm×8 mm substrate. One advantage of using an array of out-of-plane microneedles is that the resulting total cross-sectional membrane area is large enough for effective diffusion and/or delivery but the insertion of a number of out-of-plane microneedles is painless since the needles are in fact very small, actually in the micro-meter range. In addition the needle array is easy to apply by fixing (e.g., by taping) or pressing the device onto the skin rather than inserting a dialysis probe at a shallow angle several millimeter long underneath the skin.

While example detectors according to specific embodiments of the present invention are described herein as used for performing a biological assay, it will be understood to those of skill in the art that a detector according to specific embodiments of the present invention can be used in a variety of applications for detecting substances of interests. These applications include, but are not limited to: detecting contaminants in foodstuffs; detecting ripeness and/or the presence of sugars in plants or plant parts; detecting the presence of a desired substance (such as petroleum components) in an exploration operation; insuring the presence of desired compounds in a manufacturing or agricultural product, etc. Likewise, example delivery systems can be used in a variety of biological and non-biological applications.

The invention and various specific aspects and embodiments will be better understood with reference to drawings and detailed descriptions provided in this submission. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that systems and methods such as described herein can include a variety of different components and different functions in a modular fashion. Different embodiments of the invention can include different mixtures of elements and functions and may group various functions as parts of various elements. For purposes of clarity, the invention is described in terms of systems that include different innovative components and innovative combinations of innovative components and known components. No inference should be taken to limit the invention to combinations containing all of the innovative components listed in any illustrative embodiment in this specification.

In some of the drawings and detailed descriptions below, the present invention is described including various parameters of dimension and/or other parameters. These should be understood as illustrating specific and possible preferred embodiments, but are not intended to limit the invention. Many devices and/or methods have variations in one or more of the detailed parameters described herein will be apparent to persons of skill in the art having the benefit of the teachings provided herein and these variations are included as part of the present invention.

All references, publications, patents, and patent applications cited and/or provided with this submission are hereby incorporated by reference in their entirety for all purposes.

### BRIEF DESCRIPTION OF THE DRAWINGS

FIG. 1 is a schematic cross-section diagram of an example microneedle fabricating method in which a substance is deposited onto a surface with thin perpendicular pillars according to specific embodiments of the invention.

FIG. 2 is a schematic cross-section diagram of an example microneedle fabricating method showing a substance rising along pillars due to a capillary-like action according to specific embodiments of the invention.

FIG. 3 is a schematic cross-section diagram of an example microneedle fabricating method in which a substance is partially drained through a supporting plate while it adheres to pillars according to specific embodiments of the invention.

FIG. 4 is a schematic cross-section diagram of an example microneedle fabricating method in which a substance adheres to the sidewalls of the pillars while a constituent and/or reaction product of that substance out-gasses according to specific embodiments of the invention.

FIG. 5 is a schematic cross-section diagram of an example microneedle fabricating method wherein pillars that have a wider base are used to facilitate detachment from microneedles according to specific embodiments of the invention.

FIG. 6 is a schematic cross-section diagram of an example microneedle fabricating method in which a substance adheres to pillars while the pillars are lifted according to specific embodiments of the invention.

FIG. 7 is a schematic cross-section diagram of an example microneedle fabricating method showing shrinking the diameter of pillars for example using a piezoelectric force according to specific embodiments of the invention.

FIG. 8 is a schematic cross-section diagram of example microneedles after removal of pillars according to specific embodiments of the invention.

FIG. 9 is a schematic diagram of an example simplified microneedle-based system including a reservoir that can be used for a pre-filled buffer or drug delivery solution allowing transport through the microneedles according to specific embodiments of the invention.

FIG. 10 is a schematic diagram of an example microneedle-based system wherein microneedle lumens are in contact with interstitial fluids and a substance of interest is delivered or optionally diffuses through an integrated dialysis membrane with a fluid pumped past an integrated flow-through sensor according to specific embodiments of the invention.

FIG. 11 illustrates an example schematic diagram of a sensor system showing three representative microneedles, a dialysis membrane, fluid reservoirs and pumps, according to specific embodiments of the present invention.

FIG. 12 illustrates an example microneedle component with a crosslinked polymer used as a dialysis or diffusion membrane, which can be optionally functionalized with immobilized enzymes according to specific embodiments of the invention.

FIG. 13 illustrates an example device with approximately 1000 microneedles and other components according to specific embodiments of the present invention.

FIG. 14 is a schematic diagram of a skin penetration method using hollow out-of-plane microneedles according to specific embodiments of the invention.

FIG. 15 is a schematic diagram of a skin penetration method using an elastic plate with through holes according to specific embodiments of the invention.

FIG. 16 is a block diagram showing a representative example logic device in which various aspects of the present invention may be embodied.

FIG. 17 (Table 1) illustrates an example of diseases, conditions, or statuses for which substances of interest can be evaluated according to specific embodiments of the present invention.

### DESCRIPTION OF SPECIFIC EMBODIMENTS

1. Definitions

The following definitions may be used to assist in understanding this submission. These terms, as well as terms as understood in the art should be used as a guide in understanding descriptions provided herein.

A “hardenable substance” is a liquid, gel, power, mixture, condensable or sublimatable gas or other substance or material that can be shaped by one or more methods as described herein and hardened by any known curing method. Examples of hardenable substances include various polymers, metals, alloys, ceramics, cements, suspensions, solutions, mixtures, etc. Hardenable substances according to specific embodiments of the invention can be in an initially liquid or gaseous or gel or powdered form. As the fields of material science and in particular materials suitable for microfabrication and methods thereof develop, additional appropriate materials will become know that can be handled according to the methods described herein. Examples of hardenable materials that can be employed in various include epoxy, photo-curable polymer such as SU-8, metals, allows, ceramics, etc.

A “piezoelectric material” is a material that experiences a change in dimensions through the application of a voltage. Examples include various ceramics with a perovskite structure, quartz, barium titanate, lead niobate, lead zirconate titanate, Rochelle salt.

A “magnetostrictive material” is a material that experiences a change in dimensions when exposed to a magnetic field. Examples include various rare-earth elements and alloys thereof, giant magnetostrictive materials that undergo large amounts of deformations, such as alloys of terbium, dysprosium, and iron in varying compositions. Magnetostrictive material can exhibit a change in length under applied magnetism changed or a twisting which results from a helical magnetic field, often generated by passing a current through a magnetostrictive material.

2. Example Fabrication Methods

FIG. 1 is a schematic cross-section diagram of an example microneedle fabricating method in which a substance is deposited onto a surface with thin perpendicular pillars according to specific embodiments of the invention. In different embodiments, depending on the material used for the pillars and the liquid, different mechanisms are employed to make the material higher around the pins than further away from them to generate the needle shape. According to specific embodiments of the invention, a hardenable material is poured onto the molding surface from the top, enters from the sides, and/or is pushed onto this surface through bottom holes in this surface. According to other embodiments of the invention, a material can be condensated or sublimated onto a surface with micro pillars to form microneedles.

Generating Outer Needle Shape

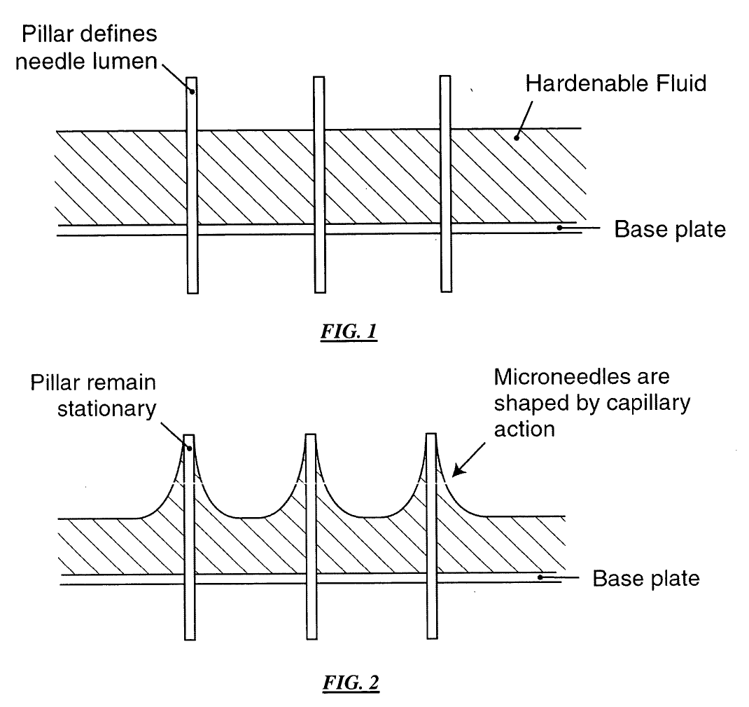

As one example embodiment, a method is illustrated in FIG. 2, wherein a capillary action causes a fluid material to rise up on the surface of the thin pillars. The height of the rise can be varied by varying a contact angle between the fluid and the pillars and/or the surface tension of the fluid and/or the specific weight of the fluid.

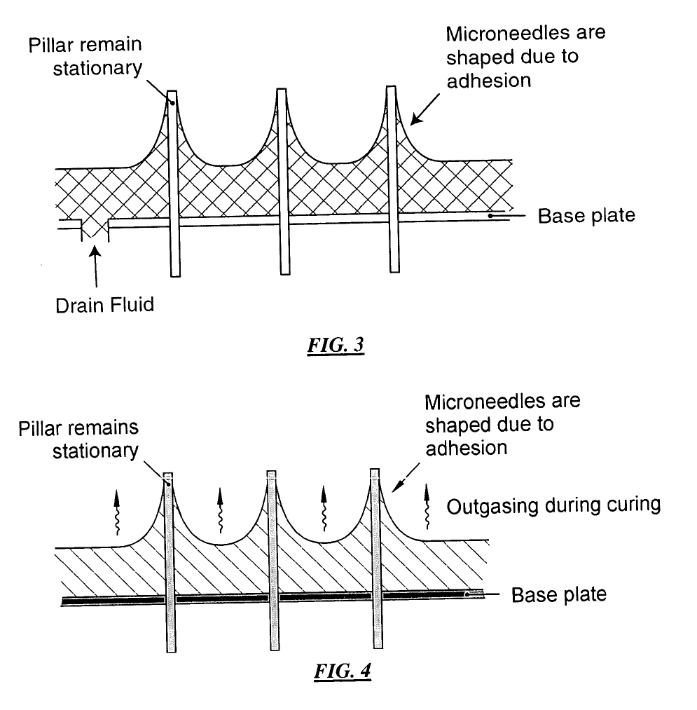

In a different embodiment, reducing material volume is used to generate a similar needle. In this case, adhesion holds a hardenable material on the sidewall of the pillars while the overall level sinks. Reducing the volume can be accomplished in a variety of different ways, according to specific embodiments of the invention. FIG. 3 is a schematic cross-section diagram of an example microneedle fabricating method in which a substance is partially drained through a supporting plate while it adheres to pillars according to specific embodiments of the invention.

In further embodiments, materials can be employed that shrink as they harden and this volume reduction effect can be used to generate needle shapes. Similarly, reduction can be accomplished by evaporation of one or more constituents or of a reaction product of a hardenable mixture or material. FIG. 4 is a schematic cross-section diagram of an example microneedle fabricating method in which a substance adheres to the sidewalls of the pillars while a constituent and/or reaction product of that substance out-gasses according to specific embodiments of the invention.

Removing Pillars to Generate Needle Lumens

In specific embodiments, pillars can be made inexpensively from materials that can easily be removed from the needles without damaging them. In specific examples, the pillars can be dissolved under conditions (e.g., temperature, solvent, ultraviolet exposure, etc.) that do not affect the optionally cured or hardened needle material, leaving the needle lumens. In this embodiment, new pillars are used for each needle fabrication.

In further embodiments, the needle structure can be removed from the pillars when the needles are not entirely hardened or after temporary softening. In this case, pillars that are wider at their base than at their tip may preferably be used to ease the pillar removal.

In a combination of these techniques, pillars are coated with a temporary or sacrificial film. After partial or complete hardening of the needles, this film is removed without damaging the pillars or needles. The resulting additional space between the needle lumens and the pillars allows easy removal of the needles from the pillars.

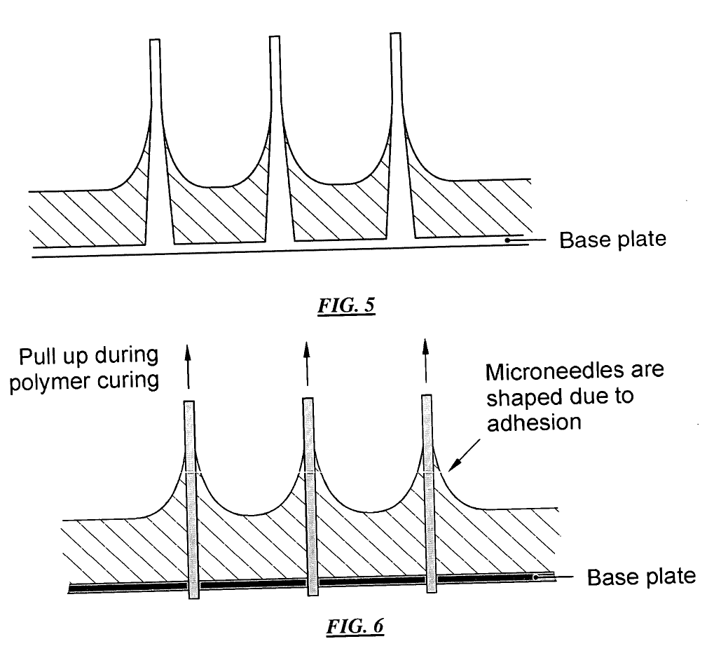

Removal of the pillars can take place either after final formation of the microneedles or during needle formation. In specific embodiments, the pillar shape can be chosen to facilitate removal of the pillars. For example, FIG. 5 is a schematic cross-section diagram of an example microneedle fabricating method wherein pillars that have a wider based are used to facilitate detachment from microneedles according to specific embodiments of the invention.

In other embodiments where the hardenable material adheres well to the pillars, the pillars can be pulled up out of the material while allowing air, another material, or more material of the pillars to follow from the base. The material will form needle shaped structures around the pillars as shown in FIG. 6.

Alternatively, a similar effect can be achieved by forcing a material, e.g., a solid, liquid or gaseous, from the flat supporting base plate up through a hardenable fluid. In this embodiment, a fluid is selected that has an adequate viscosity, and the material penetrating the liquid is drawn at a slow enough speed so that the fluid can follow and form needle shaped structures.

In further embodiments, a material can be placed onto the pillars and base through condensation or sublimation. In this embodiment, the supporting plate may be kept at a different temperature than the pillars (either colder or warmer depending on the setting characteristics of the selected hardenable substance) so that more material deposition occurs on the plate, while the material on the pillar is tapered due to a temperature gradient on the pillar surface along its axis.

Depending on the desired needle material, the optimum fabrication process for microneedles can be a combination of the concepts mentioned above.

In other embodiments, permanent pillars can be temporarily shrunk in diameter in order to facilitate needle removal. Shrinkage can be accomplished by using piezo-electric or magneto-strictive materials to make the pillars. In some embodiments, a piezo-electric or magneto-strictive material is chosen that elongates while becoming skinnier under an applied electrical or magnetic force. FIG. 7 is a schematic cross-section diagram of an example microneedle fabricating method showing shrinking the diameter of pillars for example using a piezoelectric force according to specific embodiments of the invention. Alternatively, using a very different coefficient of thermal expansion for the pillar material compared to the materials of the plate and the needles allows shrinking of the pillars relative to the remaining structures by reducing the temperature of the pillars.

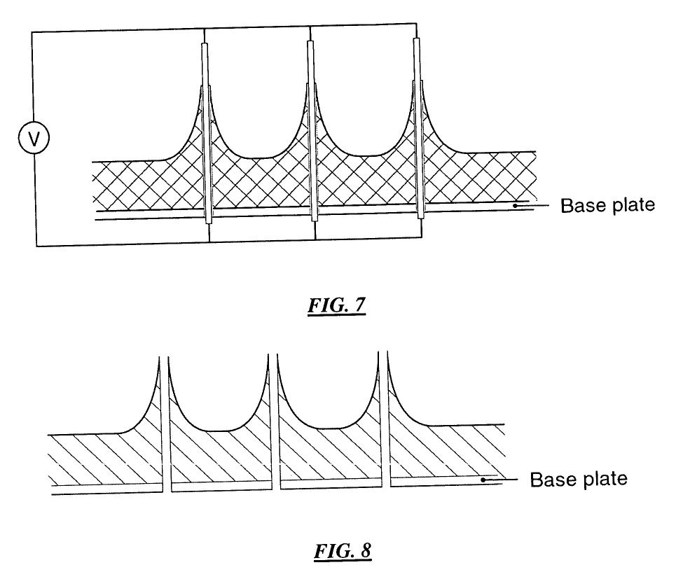

FIG. 8 is a schematic cross-section diagram of example microneedles after removal of pillars according to specific embodiments of the invention.

Curing and/or Hardening Needles

Microneedles according to specific embodiments of the invention can be further cured or hardened by a variety of known techniques, either before removal of the pillars, after removal of the pillars, or partially before and partially after. Curing or hardening can be accomplished by a change in temperature, time, evaporation, exposure to electromagnetic radiation, exposure to other materials in a gas or liquid or solid state, vibration, etc. The preferred type of hardening will depend on materials selected for forming the needles and optionally also for forming the pillars.

3. Sensors and/or Delivery Devices

Integrated systems and/or methods of the invention generally comprise an array of out-of-plane microneedles formed from a hardenable substance as described herein that are inserted into an area (such as skin) and integrated into the non-inserted side of the microneedles are components to facilitate sensing of one or more constituents from the interstitial fluid and/or delivering of substances to the interstitial fluid. The microneedles can be of various configurations, examples of which are described herein. In specific embodiments, a base plate used for forming the microneedles can be used as part of an integrated system as described herein. The additional components can include a prefilled reservoir for sensing or drug delivery. Other systems can include electronic controls, small scale or microfluidic channels, pumps, and systems, dialysis components and/or calibration components. Example configurations of such integrated systems are described in detail below. In specific embodiments, one or more of such component or attachments or channels for connecting with such components can be included in a base-plate before forming of needles from a hardenable substance as described herein.

Similar integrated systems and/or methods of the invention for substance delivery generally comprise an array of out-of-plane microneedles that are inserted into an area where a substance is to be delivered (such as skin), and integrated into the non-inserted side of the microneedles one or more channels or reservoirs for holding a substance to be delivered through the needles. In specific embodiments, a base plate used for forming the microneedles can be used as part of an integrated system as described herein. Various channels or attachment structures can be provided in the base-plate either before or after needle formation in order to ease overall system assembly.

The invention is also involved with a number of novel techniques and/or devices that enable or improve monitoring or delivery systems in particular embodiments. These techniques and/or devices have applications and uses in different systems than the examples given here, as will be understood by those of skill in the art from these teachings and in some cases are independently novel.

4. Example Integrated System Configurations

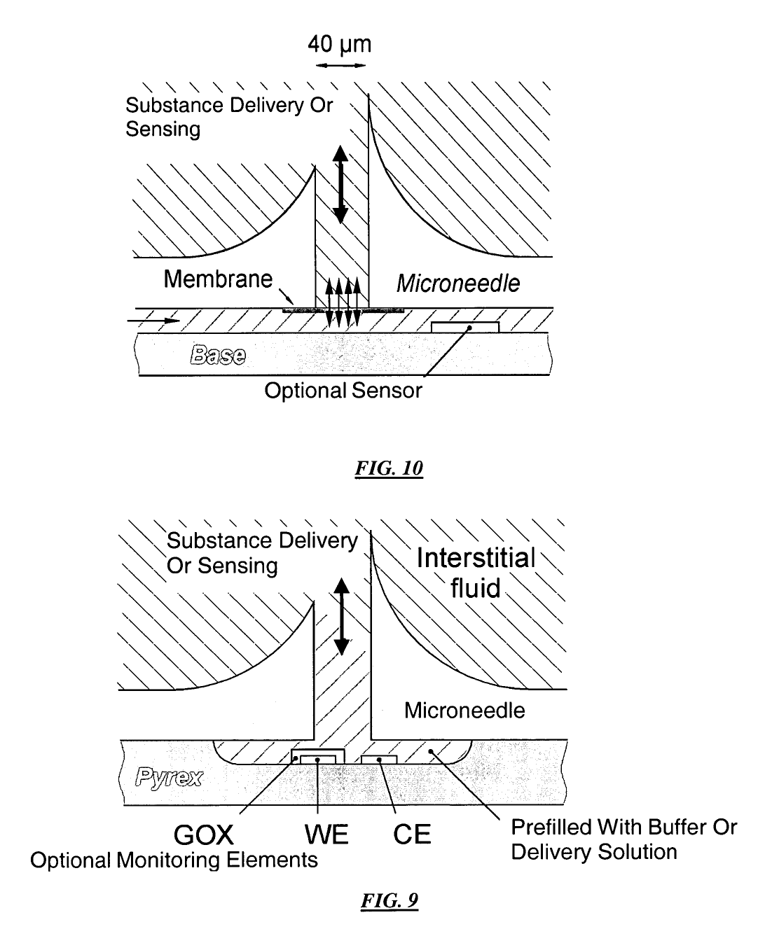

To provide different contexts for understanding embodiments of the present invention, various example embodiments of systems or portions thereof according to specific embodiments of the invention are illustrated in FIG. 9 through FIG. 12.

In each case, these figures schematically represent the combination of out-of-plane microneedle arrays such as those formed from hardenable materials as described herein with other components to form a biomedical micro system. Note that, in each of these illustrations, the one to three microneedles illustrated should be understood to represent either one single microneedle or an array of generally tens, hundreds, or a thousand or more microneedles. In some embodiments, a large set, up to all available microneedles, may be integrated with a single detection or delivery system at the base of the needle. In other systems, two or more separate detection or delivery systems can be integrated at the base of a single microneedle array, either to provide different sensing, for ease of use or manufacturing, for staged use, or to provide a control system.

FIG. 9 is a schematic diagram of an example simplified microneedle-based system including a reservoir that can be used for a pre-filled buffer or drug delivery solution allowing transport through the microneedles according to specific embodiments of the invention. For sensing, this system may not have the lifetime of reliability of dialysis-based systems in human applications. However, it is an effective basic design for prototyping and has applications where ease of manufacturing and/or reduced cost are primary considerations. It is also useful for simple drug delivery or one-time sensing systems.

Dialysis

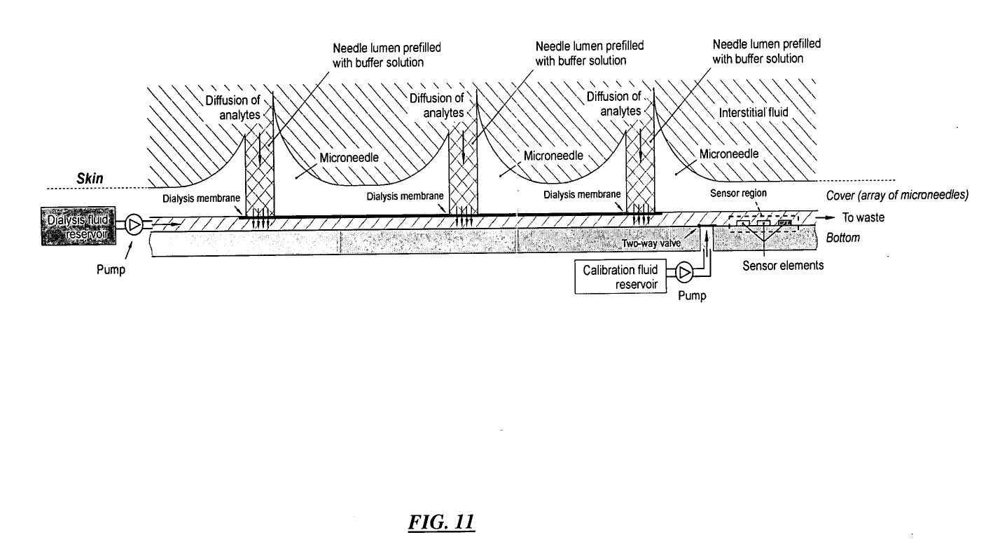

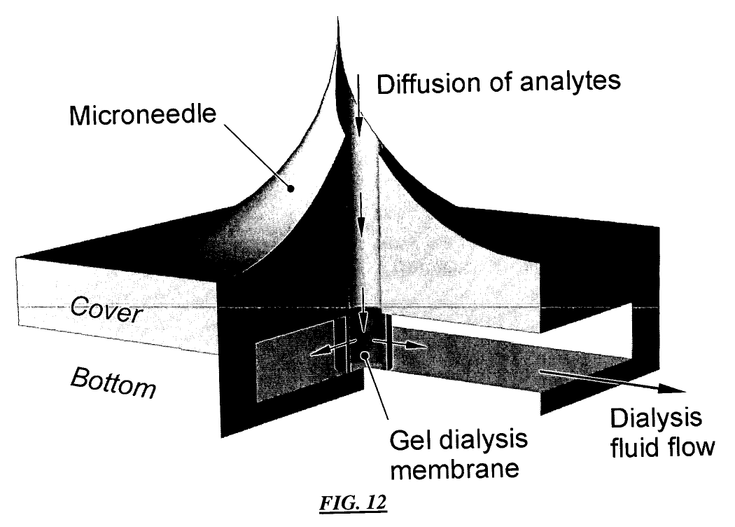

FIG. 10, FIG. 11, and FIG. 12 each illustrate different embodiments of a system that includes a dialysis membrane to separate a sensing or delivery area from the needle insertion area. One example membrane that can be used in systems according to specific embodiments of the invention is an integrated porous polysilicon dialysis membrane, as will be understood in the art. Other example membrane technology will be understood from the description herein and cited references. In systems according to specific embodiments of the invention, the dialysis membrane is any membrane or system or structure that allows diffusion of a substance and prevents one or more possibly interfering substances.

While some of these examples show optional details of sensor systems, delivery systems can be configured very similarly, including use of a dialysis membrane where desired to separate a gradually delivered substance from the area to which delivery is intended. A delivery also can be constructed by not including the dialysis membrane in the figures as illustrated, in which case the dialysis channel is used as a delivery channel.

5. Operation Examples

Operation Example Details

A system according to specific embodiments of the invention can have a number of components depending on the particular sensing or delivery application. Systems including dialysis include a dialysis barrier and can include a dialysis fluid reservoir, fluidic channels, micropumps and valves as shown. Systems including a calibration system can include a calibration fluid reservoir, fluidic channels, micropumps and valves as shown. In some embodiments, calibration fluid is segregated from sample or dialysis fluid by a moveable valve or by a flow restriction valve as shown. In alternative embodiments, calibration can be accomplished by changing the flow rate of dialysis fluid and using that fluid for calibration.

### EXAMPLE 1

FIG. 10 is a schematic diagram of an example microneedle-based system wherein microneedle lumens are in contact with interstitial fluids and a substance of interest is delivered or optionally diffuses through an integrated dialysis membrane with a fluid pumped past an integrated flow-through sensor according to specific embodiments of the invention. In this example, hollow out-of-plane microneedles formed from a hardenable substance are used to penetrate the skin and to interface with the interstitial fluid. A dialysis membrane separates the interstitial fluid and the external fluid; thus, no interstitial fluid is extracted during operation.

As an example, for the measurement of glucose concentration, dialysis fluid with a known constant glucose concentration is continuously pumped past the dialysis membrane and an integrated sensor (e.g., for glucose). Glucose diffuses through the microneedles and through the dialysis membrane into or out of the dialysis fluid. The concentration change in dialysis fluid is measured—it depends on the flow rate of the dialysis fluid and the glucose concentration in the interstitial fluid. At high flow rates (recalibrating mode) the amount of glucose diffusing into the dialysis fluid is negligible so that the glucose concentration of the dialysis fluid remains unchanged. Thus, a known concentration is measured and the sensor can be recalibrated. At low flow rates including zero flow rate (measuring mode) the concentration in the dialysis fluid changes significantly—the changed glucose concentration correlates with the glucose concentration in the interstitial fluid.

### EXAMPLE 2

FIG. 11 illustrates an example schematic diagram of a sensor system showing three representative microneedles, a dialysis membrane, fluid reservoirs and pumps, according to specific embodiments of the present invention. In this example system, separate calibration and dialysis fluid reservoirs are used, with two micropumps and valves as shown.

### EXAMPLE 3

### Microneedle with Cross-Linked Polymer

FIG. 12 illustrates an example microneedle component with a crosslinked polymer used as a dialysis or diffusion membrane, which can be optionally functionalized with immobilized enzymes according to specific embodiments of the invention. In a particular example construction, the polymer is crosslinked in the flow channel right underneath the needles where it forms walls around the needle lumen opening from the bottom to the top of this channel. In this configuration, the compounds from the interstitial fluid diffuse through the needle lumen and through the polymer wall where they might undergo enzymatic reactions before getting into the dialysis fluid in the case where enzymes have been immobilized in this membrane. In specific embodiments, using one or more of the micropillar construction techniques described above, the micropillars can be used with effectively two base plates, one forming the lower portion of the bottom section and one forming the lower portion of the needles in the cover section. The crosslinked diffusion barrier can be formed before, during, or after formation of the microneedles.

Thus, in this specific example, locally crosslinked polymer forms walls in the flow channel underneath the needles, separating the interstitial fluid from the dialysis fluid. Analytes can diffuse through this polymer.

In specific example systems, power supply and signal processing are achieved with a portable pager size device that connects to the microsystem. The portable pager size external device can also include components for connecting to a computer and/or information processing system, either through a physical adaptor or wireless connection. A wireless connected device can be used in home and or office settings to allow an individual to be remotely monitored by, for example, a health care provider or elder care provider. A large number of such monitoring devices can be used in institutional settings, such as care facilities and/or work environments and/or hospitals to monitor a number of individuals.

Integrated Systems

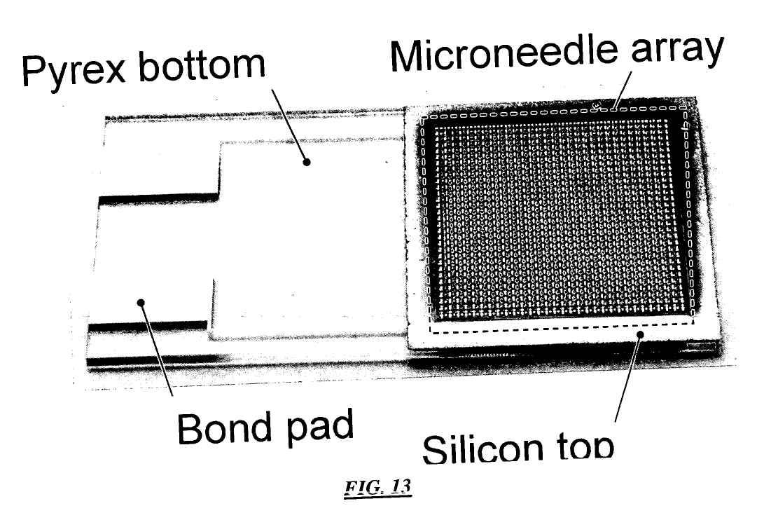

An example embodiment was fabricated using fabrication steps that will be familiar in the art in addition to the teachings provided herein and in cited references. FIG. 13 illustrates an example device with approximately 1000 microneedles and other components according to specific embodiments of the present invention. Other processes, including processing having printing, molecular growth and/or other fabrication steps as understood in the art can also be used to fabricate a device embodying the invention. Thus, FIG. 13 can also be understood as illustrating an early prototype of a simplified monitor, which only consists of out-of-plane microneedles and a glucose sensor.

6. Breaking Outer Surface or Membrane

In further embodiments, the invention involves a novel method for breaking the outer layer of mammalian skin (stratum corneum) in order to create an interface with bodily fluids. This method consists of applying a localized high pressure-load to one or multiple small location on the skin in order to yield the outer skin layer. This effect can be promoted by applying a preload to the skin in form of lateral stretcning.

Effort has been spent on generating extremely sharp microneedles, which cut the skin open in order to allow injection of fluids into the organism or sampling of bodily fluids in the same fashion as in the case of hypodermic needles. However, fabrication of extremely sharp small needles can be difficult and expensive. Furthermore, it is unclear if the sharp tips of these microneedles have a sufficient mechanical strength to prevent breakage during usage. In addition, the skin and the underlying tissue are very flexible for small deflection as typically caused by short microneedles, so that the classical approach of cutting through the stratum corneum risks to fail due to insufficient contact pressure. This problem is even more severe in the case of needle arrays, where a distributed load over a wide area of skin can result in a bed of nails effect, which merely leads to uniformly pushing down the skin. Nevertheless, microneedles allow easy integration into advanced drug delivery systems or into systems for detection of body fluids and/or compounds in an organism.

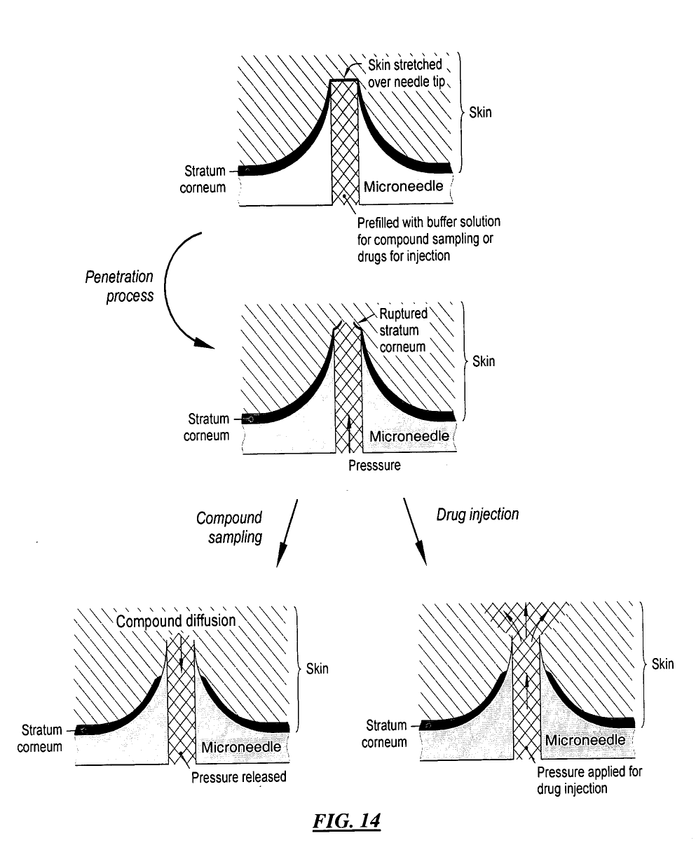

This mechanism can be used for sampling or delivery through the short microneedles. In this approach, the outer skin layer can be broken by applying high pressure to a small local skin region, which results in rupture of the cell matrix. This effect can be promoted by applying a preload to the skin in form of lateral stretching. Pressing such a microneedle against the skin as shown in FIG. 14 (top) stretches the skin over the needle tip, so that additional pressure applied to the fluid inside the needle lumen results in yielding of the skin, which ruptures and opens a passage way between fluids inside the needle lumen and bodily fluids underneath the broken skin layer, FIG. 14 (middle). The stratum corneum slips back while the needle tip is inserted into the to epidermis.

This opened passage can be used for multiple purposes. Compounds or fluids from within the organism can get transported through the needle lumen by diffusion or other transport mechanisms as shown in FIG. 14 (bottom left), so that these compounds can be detected or quantified for monitoring purposes. Such compounds or fluids could be glucose, lactate, proteins, lipids, DNA, cells or blood.

This flow passage can also be used for injection of fluids into the organism as shown in FIG. 14 (bottom right). In addition, this interface with bodily fluids can be used to send and/or collect electrical or optical signals into or from the organism for detection purposes. Multiple needles in form of an array can be used simultaneously for an identical purpose or multiple applications.

As a major advantage, this perforation method does not require extremely sharp microneedles, which allows simpler fabrication at low cost, for example using hardenable substances as described herein. Furthermore, less sharp microneedles are less susceptible to breakage of their tip increasing their reliability. In addition, the usage of less sharp needles is safer since they only penetrate skin in response to the combined forces of stretching the skin and pressurizing the fluid.

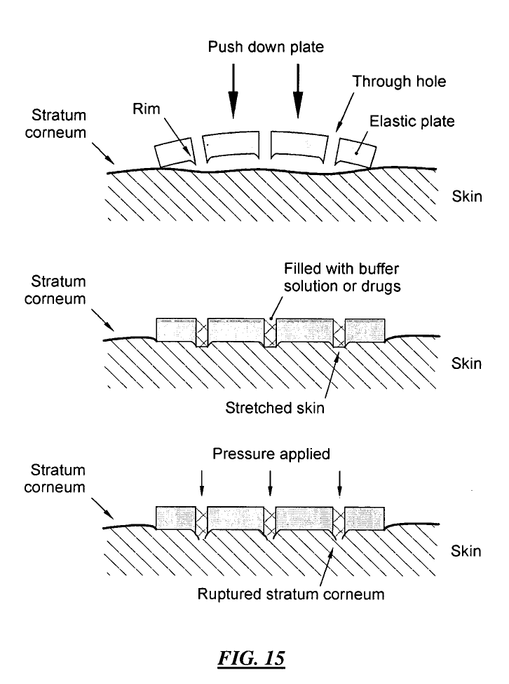

In certain cases this method of skin perforation can be enhanced using structures formed from hardenable materials that are flexible after manufacture. FIG. 15 shows an apparatus that stretches the skin as it is being pressed against it. The base of this apparatus extends laterally while its edges hold on to the skin. This base also provides small trough holes, which can be used to apply additional pressure to the small regions of the skin underneath these holes by pressurizing a fluid from the side of the base opposite to the skin. Small rims around these openings on the side of the skin provide a good seal between the apparatus and the skin during pressure application.

As can be seen in the figure, this method is enhanced by use of a flexible push down plate or and needles or other sharp structures that can be pushed against the skin and deformed to some extent outwards. According to specific embodiments of the invention, such a device can be made using one or more methods for forming microneedle-like structures from hardenable materials as described above.

7. Diagnostic Uses

As described above, following identification and validation of a sensor for a particular substance, including biological molecules such as sugars, proteins, fats, or any substance of interest according to the invention, in specific embodiments such detectors are used in clinical or research settings, such as to predictively categorize subjects into disease-relevant classes, to monitor subjects on a continuous basis to detect a substance of interest, etc. Detectors according to the methods the invention can be utilized for a variety of purposes by researchers, physicians, healthcare workers, hospitals, laboratories, patients, companies and other institutions. For example, the detectors can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine most appropriate additional diagnostic testing for the patient, among other clinically and epidemiologically relevant applications. Essentially any disease, condition, or status for which a substance or difference can be detected in an interstitial fluid can be evaluated, e.g., diagnosed, monitored, etc. using the diagnostic methods of the invention, see, e.g. Table 1. Essentially any disease, condition, or status for which a substance can be delivered to effect treatment to interstitial fluid can be treated, using the diagnostic methods of the invention, see, e.g. Table 1.

In addition to assessing health status at an individual level, the methods and diagnostic sensors of the present invention are suitable for evaluating subjects at a “population level,” e.g., for epidemiological studies, or for population screening for a condition or disease.

### Web Site Embodiment

The methods of this invention can be implemented in a localized or distributed data environment. For example, in one embodiment featuring a localized computing environment, a sensor according to specific embodiments of the present invention is configured in proximity to a detector, which is, in turn, linked to a computational device equipped with user input and output features. In a distributed environment, the methods can be implemented on a single computer, a computer with multiple processes or, alternatively, on multiple computers. Sensors according to specific embodiments of the present invention can be placed onto wireless integrated circuit devices and such wireless devices can return data to a configured information processing system for receiving such devices. Such devices could, for example, be configured to be affixed to a subject's body.

Kits

A detector according to specific embodiments of the present invention is optionally provided to a user as a kit. Typically, a kit of the invention contains one or more sensors constructed according to the methods described herein. Most often, the kit contains a diagnostic sensor packaged in a suitable container. The kit optionally further comprises an instruction set or user manual detailing preferred methods of using the kit components for sensing a substance of interest.

When used according to the instructions, the kit enables the user to identify disease or condition specific substances (such as sugars and/or fats and/or proteins and/or antigens) using patient tissues, including, but not limited to interstitial fluids. The kit can also allow the user to access a central database server that receives and provides information to the user. Additionally, or alternatively, the kit allows the user, e.g., a health care practitioner, clinical laboratory, or researcher, to determine the probability that an individual belongs to a clinically relevant class of subjects (diagnostic or otherwise).

### Embodiment in a Programmed Information Appliance

The invention may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

Integrated Systems

Integrated systems for the collection and analysis of detection results, including detection or expression profiles, molecular signatures, as well as for the compilation, storage and access of the databases of the invention, typically include a digital computer with software including an instruction set for sequence searching and/or analysis, and, optionally, one or more of high-throughput sample control software, image analysis software, data interpretation software, robotic or fluidic controls for transferring solutions from a source to a destination (such as a detection device) operably linked to the digital computer, an input device (e.g., a computer keyboard) for entering subject data to the digital computer, or to control analysis operations or high throughput sample transfer by the robotic control armature. Optionally, the integrated system further comprises an electronic signal generator and detection scanner for probing a needle array. The scanner can interface with analysis software to provide a measurement of the presence or intensity of the hybridized and/or bound suspected ligand.

Readily available computational hardware resources using standard operating systems can be employed and modified according to the teachings provided herein, e.g., a PC (Intel x86 or Pentium chip-compatible DOS™, WINDOWS™, LINUX™, or Macintosh, Sun or PCs will suffice) for use in the integrated systems of the invention. Current art in software technology is adequate to allow implementation of the methods taught herein on a computer system. Thus, in specific embodiments, the present invention can comprise a set of logic instructions (either software, or hardware encoded instructions) for performing one or more of the methods as taught herein. For example, software for providing the described data and/or statistical analysis can be constructed by one of skill using a standard programming language such as Visual Basic, Fortran, Basic, Java, or the like. Such software can also be constructed utilizing a variety of statistical programming languages, toolkits, or libraries.

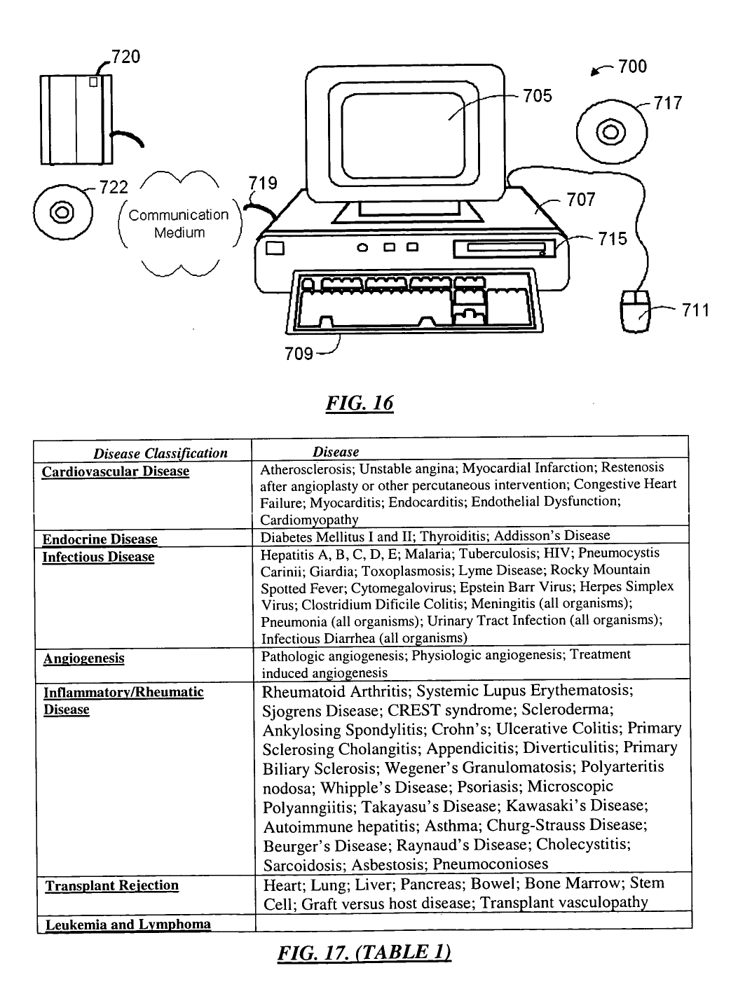

FIG. 16 is a block diagram showing a representative example logic device in which various aspects of the present invention may be embodied. FIG. 16 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection.

Various programming methods and algorithms, including genetic algorithms and neural networks, can be used to perform aspects of the data collection, correlation, and storage functions, as well as other desirable functions, as described herein. In addition, digital or analog systems such as digital or analog computer systems can control a variety of other functions such as the display and/or control of input and output files. Software for performing the electrical analysis methods of the invention is also included in the computer systems of the invention.

Thus, a microneedle-based system according to specific embodiments of the invention can be employed as an effective monitoring or delivery microneedle array system. Due to the optimum needle dimensions, it is sufficient to simply press the system onto the skin in order to reach the desired location in the epidermis with an abundant amount of interstitial fluid. The nerve endings are located deeper in the skin so that this procedure is painless. The glucose monitor can be attached to a skin location (for example, with a self-adhesive, medical tape, a band, etc.) by the patient himself without an assisted insertion procedure.

### Other Embodiments

Although the present invention has been described in terms of various specific embodiments, it is not intended that the invention be limited to these embodiments. Modification within the spirit of the invention will be apparent to those skilled in the art. It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this submission, including any references filed as part of an Information Disclosure Statement, are incorporated by reference in their entirety.

What is claimed:

1. A method of manufacturing a microneedle device, comprising:
providing a substantially planar base plate;
providing a plurality of micropillars substantially perpendicular to said base plate;
introducing a hardenable substance onto said base plate;
selecting conditions such that said substance is retained with a greater circumference near to said base plate and extends up said pillars to a lesser circumference to create a needle shape; and
removing said pillars while preserving said needle shape;
thereby forming a plurality of hollow microneedles.

2. The method of claim 1 further wherein:
said base plate having a plurality of holes; and
said plurality of micropillars able to be inserted and/or removed from said holes.

3. The method of claim 1 further wherein:
said base plate is substantially rigid.

4. The method of claim 1 further wherein said micropillars are substantially cylindrical in shape.

5. The method of claim 1 further wherein said micropillars are substantially conical in shape.

6. The method of claim 1 further wherein said introducing is accomplished by one or more of:
pouring said substance onto said base plate;
introducing said substance from sides of said base plate;
introducing said substance from below said base plate;
condensing said substance onto said base plate; and
sublimating said substance onto said base plate.

7. The method of claim 6 further wherein said base plate is kept colder than the pillars so that more material deposition occurs on the plate while material on the pillar is tapered due to a temperature gradient on the pillar surface along its axis.

8. The method of claim 1 further wherein said hardenable substance is selected from the group consisting of:
a polymer;
a melt;
a powder;
a solution; and
a suspension.

9. The method of claim 1 further wherein said conditions comprises one or more of:
choosing pillar surface material and substance properties so as to cause said substance to rise around said pillars by capillary-type action to define a shape of said needles; and
altering a temperature at said base plate to cause more of said hardenable substance to remain nearer said base plate.

10. The method of claim 9 further wherein:
a height of rise by capillary action is modified by selecting one or more of:
a contact angle between the substance and the pillars;
surface tension between the substance and/or pillar surface and/or baseplant surface: and
specific weight of the substance.

11. The method of claim 1 further wherein said conditions comprises controlling the contact time of said substance to said pillars and said base plate to form said needle shape.

12. The method of claim 11 further wherein said controlling comprises one or more of:
gradually draining said substance while some of said substance adheres to pillars, such that more of said substance adheres near to said base plate;
gradually introducing said substance while some of said substance adheres to pillars, such that more of said substance adheres nearer to said base plate;
selecting for said substance a material that shrinks as it cures;
evaporation of one or more constituents of said substance.

13. The method of claim 1 further wherein pillars are removed after or during hardening of said substance leaving passages and forming needle lumens.

14. The method of claim 13 further wherein pillars are removed by one or more of:
dissolving under conditions that do not adversely affect said needles;
removing pillars from said needles when said needles are not entirely hardened or after temporary softening.

15. The method of claim 1 further wherein said pillars are wider at their base than at their tip.

16. The method of claim 1 further comprising:
coating said pillars with a sacrificial film;
after partial or complete hardening of the needles, removing said film without damaging said pillars or said needles.

17. The method of claim 13 further wherein pillars are removed by one or more of:
temporarily shrinking a diameter of said pillars using a piezo-electric action of said pillars;
temporarily shrinking a diameter of said pillars using a magneto-strictive action of said pillars;
shrinking a diameter of said pillars with respect to said needles by reducing temperature of said pillars and selecting a material for said pillars with a different coefficient of thermal expansion for the pillar material compared to the hardened microneedle material.

18. The method of claim 13 further wherein pillars are pulled up out of the substance while allowing air, another material, or more material of the pillars to follow from the base to cause the substance to form needle shaped structures around the pillars.

19. A method of manufacturing a microneedle array, comprising:
providing a substantially planar base plate;
providing a plurality of microholes in said base plate;
introducing a hardenable substance onto said base plate;
selecting conditions such that said substance is retained with a greater circumference near to said base plate; and
forcing a solid, liquid or gaseous material from the flat supporting base plate up through the substance wherein the substance is selected that has an adequate viscosity, and the material penetrating the substance is drawn at a slow enough speed so that the substance can follow; and
thereby forming a plurality of hollow microneedles.

20. The method of claim 1 further wherein said microneedles are further hardened by one or more additional techniques including:
applying a temperature appropriate for hardening a selected material;
removing a volatile solvent;
applying one or more curative agents appropriate for hardening a selected material; and
applying vibrations or other mechanical forces appropriate for hardening a selected material.

21. The method of claim 1 further comprising:
providing fluidic channels and or reservoirs proximal to a non-insertive side of said microneedles such that when said microneedles are pressed against a surface of interest operative fluidic contact to a region behind said surface allows sensing or delivery of substances of interest.

22. The method of claim 1 further comprising:
placing a dialysis membrane proximal to a non-insertive side of said microneedles;
providing a reservoir for a fluid in contact with a second surface of said dialysis membrane;
such that when said microneedles are pressed against a surface of interest, one or more substances of interest can pass through said dialysis membrane.

23. The method of claim 22 further comprising:
providing one or more sensors in contact with said fluid for measuring and/or detecting one or more substances of interest.

24. The method of claim 21 further wherein:
said plurality comprises at least 8 microneedles.

25. The method of claim 21 further comprising:
said plurality comprises at least 200 microneedles.

26. The method of claim 21 further comprising:
said plurality comprises at least 750 microneedles.

27. The method of claim 21 further wherein:
said microneedles are between about 100 micrometers and about 300 micrometers long.

28. A device monitoring one or more substances of interest comprising:
a plurality of out-of-plane microneedles formed from a hardenable material for applying to a surface of an internal region, said microneedles long enough to sample one or more substances of interest at and/or just below said surface;
said microneedles comprising one or more membranes on a side opposite a side applied to said surface such that said membrane is not placed under said surface;
said membrane separating said microneedles from a dialysis material;
such that dialysis occurs outside of said internal region.

29. The device of claim 28 further wherein:
said one or more dialysis membranes comprise a large total membrane surface that can remain outside of said internal region.

30. The device of claim 28 further wherein:
a plurality of said microneedles are pre-filled with a fluid before said applying.

31. A method of monitoring or delivering substances of interest to an internal region comprising:
applying a plurality of microneedles formed from a hardenable substance to a surface of an internal region, said microneedles long enough to prestress a region of the surface at a needle lumen;
applying high pressure to a small local surface region through said microneedles to cause rupture of the surface to open a connection between fluids inside the needle lumen and fluids underneath the broken surface layer; and
using said connection monitor or deliver one or more substances of interest at and/or just below said surface.

32. A method of monitoring or delivering substances of interest to an internal region comprising:
applying a plurality of puncture structures containing through-holes formed from a hardenable substance with a flexible backing to a surface of an internal region, said puncture structures long enough to prestress a region of the surface at a through-hole;
applying a deforming force to a flexible backing of said structures, said force widening a through-hole area in contact with said prestressed region to cause rupture of the surface to open a connection between fluids inside the through-hole and fluids underneath the surface layer; and
using said connection monitor or deliver one or more substances of interest at and/or just below said surface.
